(19) United States
(12) Patent Application Publication
(10) Pub. No.: US 2021/0308144 A1
(43) Pub. Date: Oct. 7, 2021

(54) Synthesis of New Potent Aromatase Inhibitors Through Biocatalysis of Anti-Cancer Drugs, Atamestane, Drostanolone Enanthate, and Exemestane

(71) Applicants: Atia-tul- Wahab, Karachi (PK); Muhammad Iqbal Choudhary, Karachi (PK); Mahwish Siddiqui, Karachi (PK); Nimra Naveed Shaikh, Karachi (PK); Nisha Khan, Karachi (PK); Atta-ur- Rahman, Karachi (PK)

(72) Inventors: Atia-tul- Wahab, Karachi (PK); Muhammad Iqbal Choudhary, Karachi (PK); Mahwish Siddiqui, Karachi (PK); Nimra Naveed Shaikh, Karachi (PK); Nisha Khan, Karachi (PK); Atta-ur- Rahman, Karachi (PK)

(21) Appl. No.: 17/199,481

(22) Filed: Mar. 12, 2021

(51) Int. Cl. A61K 31/566; A61K 31/567

(52) U.S. Cl. CPC A61K 31/566; A61K 31/567

(57) ABSTRACT

New analogues of anti-cancer drugs atamestane (1), drostanolone enanthate ((3), and exemestane (6) were synthesized through biotransformation. New derivatives, 14α-hydroxy-1-methylandrosta-1,4-diene-3,17-dione ((2) (IC₅₀, 9.7±0.72 nM) of 1 (IC₅₀, 13.8±0.2 nM), and 2-methylandrosta-12β,17β-dihydroxy-1,4-diene-3-one (4) (IC₅₀, 4.23±0.133 nM) of 3 (IC₅₀, 6.4±0.06 nM) showed a potent inhibition against human aromatase enzyme and thus have the potential to treat ER+ breast-cancers and other related diseases. New metabolites, 2α-methyl-9α,17β-dihydroxy-5α-androstan-3-one ((5) (IC50=793.0±29.9 nM) of 3, 6-methylene-3α,7β,17β-trihydroxy-5β-androstane (7) (IC₅₀, 46.1±0.81 nM), and 11α,17β-dihydroxy-6-methylene-androsta-1,4-diene-3-one (8) (IC_{5O}=12797.0±844 nM) of exemestane (6) (IC₅₀=232.0±31 nM) also showed a remarkable anti-aromatase activity. Aromatase is an enzyme, involves in the synthesis of estrogen (ER). Increased amount of ER due to overexpression of aromatase in the body, promotes cancerous cells growth in breast. Therefore, aromatase enzyme is a key target for the discovery of chemotherapeutic agents against ER+ breast-cancers.

### BACKGROUND OF THE INVENTION

Breast cancer is a leading cause of deaths in women, affecting over a million females annually worldwide. In general, two-thirds of breast cancers are hormone dependent, i.e., require estrogens for their growth. Aromatase enzyme, an estrogen synthase, is a member of P450 cytochrome system. This catalyzes the transformation of androgen to estrogen through aromatization of steroidal ring “A”. Estrogen plays important roles in the development of female organs, and regulation of reproductive system. Presence of high amount of estrogen hormone in postmenopausal females is directly linked with the increased risk of breast cancers. Such types of breast cancers are normally treated by inhibiting the aromatase enzyme in order to block the production of estrogens in the body. Therefore, aromatase enzyme is one of the most promising targets for the treatment of ER+ (estrogen-responsive) breast cancers. Aromatase inhibitors have also been reported to possess anti-estrogens property, and there are, used for the treatment of disorders due to the imbalance between estrogen and androgen levels. Currently, a number of steroidal, and non-steroidal aromatase inhibitors are in clinical use, but due to their lower selectivity and efficacy, they are inadequate for the treatment of breast cancers. Aromatase enzymes is normally present in placenta, and granulosa cells of ovarian follicles [Ghuge et al., Curr. Enzym Inhib. 2020, 16, 45; Waks and Winer, JAMA 2019, 321, 288; Sun et al., Int. J. Biol. Sci. 2017, 13, 1387; DeSantis et al., CA Cancer J. Clin. 2016, 66, 290; Ghoncheg et al., Pac. J. Cancer, Prev. 2015, 16, 6081; Brueggemeier et al., Endocr. Rev. 2005, 26, 331],

Many steroids either natural, synthetic or semi-synthetic, have effectively been used for the treatment of breast cancers since long. However, their insufficient efficacy, and several adverse effects on normal cells, make them sub-optimal treatment options,

Atamestane (1), a synthetic steroidal anti-cancer drug SH-489) (under development), is reported as a potent aromatase inhibitor, which blocks the estrogen production in the body, and prevents the growth of cancer cells in the breast. It has the potential to treat estrogen-dependent breast cancers, either alone or in combination with toremifene [Goss et al., J. Natl. Cancer Inst. 2005, 97, 1262: Kuhnz et al., Eur. J. Drug Metab. Ph. 1994, 19, 137].

Drostanolone enanthate (3) is DHT (dihydrotestosterone)-derived anabolic steroidal drug, used by athletes to strengthen their muscles without gaining weight. In addition, drostanolone propionate (brand, Masteron Propionate), and drostanolone enanthate (3) (brand, Masterone Enanthate) have the ability to inhibit the estrogen production, and act as potent aromatase inhibitors. Therefore, they are used for the treatment of breast cancer [Choudhary et al., Front. Pharmacol. 2017, 8, 900; Chowdhury et al., Clin. Oncol. 1976, 2, 203; Marinov et al., Khirurgiia, 1986, 40, 80],

Exemestane (6) is the most commonly used anti-cancer steroidal drug, which is marketed under the brand name of Aromasin. It has been reported to possess a good ability to lower the estrogen level, and increase the testosterone and ICIF (insolin-like growth factor) levels. This makes exemestane (6) an effective medication for the treatment of ER+ breast cancers [Scott and Wiseman, Drug, 1999, 58, 675; Baydoun et al., Chem. Cent. J. 2013, 7, 1].

Synthesis of structural analogues of steroidal drugs is a challenging, and demanding task. This typically requires expensive and toxic reagents, and catalysts. Their derivatization can produce more potent compounds with better pharmacodynamic profile, as compared to the parent drugs, Biotransformation is an effective, and robust method to synthesize compounds that resemble to substrates. It is efficiently applied, where conventional schemes are difficult, as it is arbitrated by low-cost, coo-friendly, and selective biocatalysts. Often whole-cells, such as bacteria, fungi, yeast, and plant cells, are used as biocatalysts. The technique is successfully employed to bring structural modifications in almost all classes of organic compounds [Siddiqui et al., J. Adv. Res. 2020, 24, 69: Alcantara and Alcantara, Biocatal, Biotransfor. 2018. 36, 12; Atia-tul-Wahab et al., Bioorg. Chem. 2018, 77, 152; Choudhary et al., Front. Pharmacol. 2017, 8, 900; Siddiqui et al., PloS One, 2017, e0171476; Bianchini et al., Front. Microbiol. 2015, 6, 1433].

### BRIEF SUMMARY OF THE INVENTION

In continuation of our fungal-catalyzed structural modifications of bioactive steroids [Siddiqui et al., J. Adv. Res. 2020, 24, 69; Ibrahim et al., Steroids, 2020, 162, 108694; Hussain et al., RSC Advances, 2020, 10, 451; Atia-tul-Wahab et al., Bioorg. Chem. 2018, 77, 152; Choudhary et al., Front. Pharmacol. 2017, 8, 900; Siddiqui et al., PloS One, 2017, e0171476; Bano et al., Steroids, 2016, 112, 168], and based on reported anti-cancer activity of drugs 1, 3, and 6, we have focused on their whole-cell fungal catalyzed structural modifications,

Biotransformation of atamestane (1) with Fusarium lini yielded a new metabolite, 14α-hydroxy-1-methylandrosta-1,4-diene-3,17-dione (2).

Biotransformation drostanolone enanthate (3) with Glomerella fusarioides afforded two new derivatives, 2-methylandrosta-12β,17β-dihydroxy-1,4-diene-3-one 4), and 2α-methyl-9α,17β-dihydroxy-5α-androstan-3-one (5).

Similarly, two new compounds, 6-methylene-3α,7β,17β-trihydroxy-5β-androstane (7) and 11α,17β-dihydroxy-6-methylene-androsta-1,4-diene-3-one (8) were obtained from biotransformation of exemestane (6) with Glomerella Fusarioides.

Moreover, two new analogues, 11β,17β-dihydroxy -7α,17α-dimethyl-estra-1,3,5-triene-3-one (10) and 17β-Hydroxy-7α,17α-dimethylester-4,6-diene-3-one (11) were obtained from the biotransformation of mibolerone (9).

Based on reported anti-aromatase activity of drugs 1,3, and 6, their analogues 2, 4-5, and 7-8 were evaluated against human placental microsomes (aromatase enzyme). New derivative 2 (IC₅₀=9.7±0.72 nM) showed a potent activity against human aromatase, as compared to parent drug atamestane (1) (IC₅₀=13.8±0.2 nM), and standard drug exemestane (6) (IC₅₀=232±31 nM). Similarly, new metabolite 4 (IC₅₀=4.23±0.133 nM) was also identified as a potent aromatase inhibitor in comparison to parent drug, drostanolone enanthate (3) (IC₅₀=6.4±0.06 nM), and standard drug exemestane (6). Derivative 7 (IC₅₀=46.1±0.81 nM) also showed a remarkable inhibition against human, aromatase Compounds 5 (IC₅₀=793±29.9 nM), and 8 (IC₅₀=12797±844 nM) also showed a significant inhibition potential against human aromatase enzyme.

New metabolites 2, 4-5 and 7-8 were identified as non-cytotoxic against BJ (human fibriblast) cell line.

### BREIF DESCRIPTION OF THE DRAWINGS

FIG. 1 depicts the structures of atamestane (1), and its new metabolite 2 via mediated transformation of drug 1, along with their aromatase inhibition, and cytotoxicity against human fibroblast (BJ) cell line.

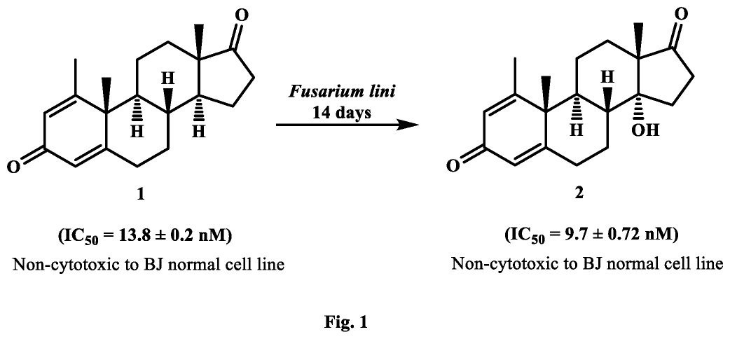

FIG. 2 depicts the structures of drostanolope enanthate (3) and its new metabolites 4-5 via G. fusarioides-mediated transformation of drug 3, along with their aromatase inhibition, and cytotoxicity against human fibroblast (BJ) cell line.

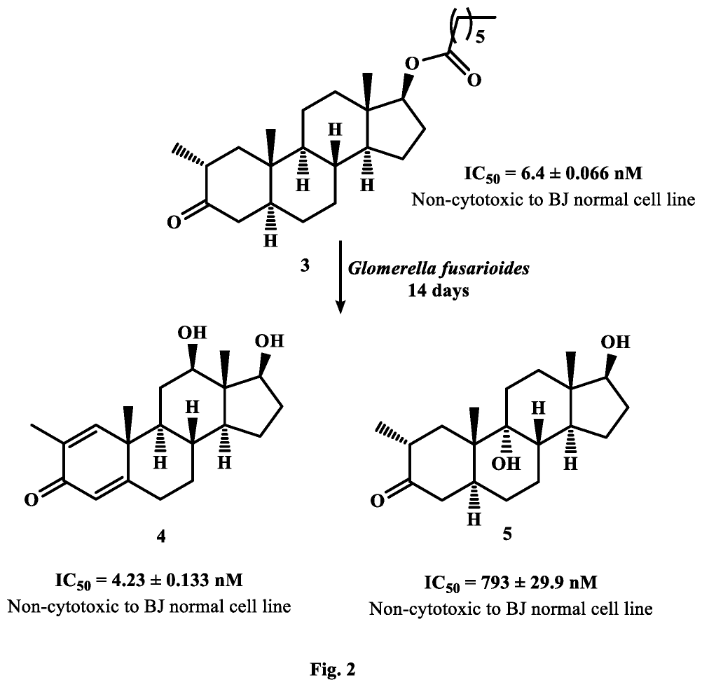

FIG. 3 depicts the structures of exemestane and its new metabolites 7-8 via G. fusarioides-mediated transformation of drug 6, along with their aromatase inhibition, and cytotoxicity against human fibroblast (BJ) cell line.

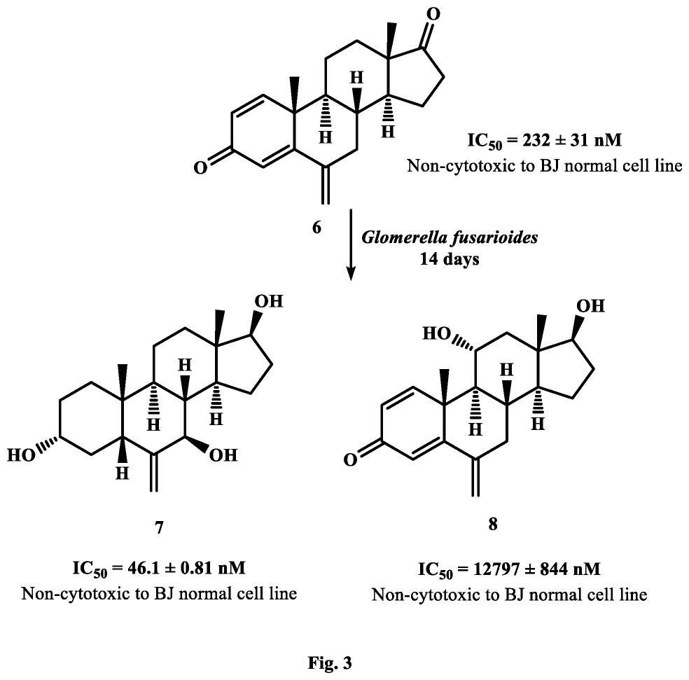

### DETAILED DESCRIPTION OF THE INVENTION

### Experimental

### Media Preparation

One-liter media for each fungus was prepared by mixing 10 g glucose, g NaCl, 5 g peptone, 5 g KH₂PO₄, and 10 mL glycerol in 1 L distilled water.

### Fermentation

On the basis of small-scale screening results, 5 L of media for each fungus was prepared by mixing aforementioned ingredients. Media (200 mL) was transferred into 25 Erlenmeyer flasks of 500 mL and cotton plugged. These flasks were autoclaved at 121° C., and then cooled at room temperature. Media was inoculated with each fungal cell culture. separately under sterilized conditions, and placed for four days on a rotary shaker (121 rpm). After the mature growth of F. lini, and G. fusarioides in each flask, 2 g of each drug was dissolved in 25 mL of methanol, and dispensed (1 mL) in each fungal-containing flask. These flasks were again placed on rotary shaker (121 rpm) at 25° C.

### Extraction

After incubation, ethyl acetate (EtOAc) was added in each flask to stop the reaction, and filtered to separate biomasses. Each filtrate was extracted with 25 L of EtOAc separately. Each extract (organic layer) was made moisture free by adding anhydrous sodium sulfate (Na₂SO₄), filtered, and evaporated through a rotary evaporator.

### Isolation and Purification

Column chromatography (CC) was performed to fractionate each crude extract (1-3), using a mobile phase of hexanes-acetone (with 5-100% gradients of acetone). One fraction from crude 1, two fractions from crude 2, and two fractions from crude 3 were obtained. Compound 2 (H₂O-ACN, 3/7; Rₜ=26 min) from fraction 1, compounds 4 (H₂O-ACN, 3/7; Rₜ=19 min), and 5 (H₂O-ACN, 3/7; Rₜ=21 min)) from crude 2, and compounds 7 (1-170-ACN, 4/6; Rₜ=22 min), and 8 (H₂O-ACN, 4/6; Rₜ=24 min) from crude 3, were purified through recycling RP-HPLC.

14α-Hydroxy-1-methylandrosta-1,4-diene-3,17-dione (2)

White solid; UVλₘₐₓ (log ε): 249 (5.78); melting point: 171-173° C.; [α]_{D}²⁵=−126.6 (c 0.001); HREI-MS m/z 314.1873 [M]⁺ (calc. 314.1882) (C₂₀H₂₆O₃); EI-MS (%): 314.3 [M]⁺ (10.8); IR υₘₐₓ (cm⁻¹): 3412 (O—H), 2934 (C—H), 1737 (C═O), and 1655 and 1604 (C═C—C═O); ¹H NMR (δ) (d₆-acetone), H-2 (6.06, s), H-4 (5.98, s), H₂-6 (2.74, in; 2.41, overlap); H₂-7 (2.01, overlap; 1.54, overlap). H-8 (2.20, overlap), H-9 (1.68, overlap), H₂-11 (1.83, overlap; 1.69, overlap), H₂-12 (2.02, overlap, 2[H]), OH-14 (3.53, s), H₂-15 (1.82, overlap; 1.42, overlap), H₂-16 (2.33, overlap; 2.18, overlap), H₃-18 (1.04, s), H₃-19 (1.42, s), H₃-20 (2.14, s), ¹³C-NMR (δ) (d₆-acetone), C-1 (166.3), C-2 (129.6), C-3 (185.3), C-4 (124.0), C-5 (170.2), C-6 (32.9), (29.1), C-8 (38.6) C-9 (52.5.), C-10 (47.6), C-11 (24.4), C-12 (30.5), C-13 (52.7), C-14 (80.4), C-15 (25.5), C-16 (33.2), C-17 (217.4), C-18 (17.8) C-19 (16.2), C-20 (23.6)

2-Methylandrosta-12β,17β-dihydroxy-1,4-diene-3-one 4)

White solid; UV (log ε); 251 (5.69); melting point: 235-237° C.; UV λₘₐₓ: 248 nm (log ε 6.8); [α]_{D}²⁵=−27 (c 0.001); IR υₘₐₓ (cm⁻¹); 3322 (OH), 1733 (C═O), 1664, 1624 (C═C—C═O); HRFAB-MS (+ve mode) m/z 317.2124 [M+H]⁺ (calc. 317.2117) (C₂₀H₂₉O₃); FAB-MS (−ve mode) m/z 315.2; ¹H-NMR (δ) (CDCl₃), H-1 (6.18, s) H-4 (6.07, s), H₂-6 (2.57,m; 2.35, m), H₂-7 (1.99, m; 1.82, m), H₂-8 (1.66, overlap), H-9 (1.15, m), H₂-11 (1.74, m; 0.96, in), H-12 (3.43, m), H-14 (0.84, m), H₂-15 (1.63, overlap; 1.43, m), H₂-16 (2.04, m; 1.48, m), H-17 (3.83, t, J_{17,16}=8.7 Hz), H₃-18 (0.85, s), H₃-19 (1.33, s), H₃-20 (2.11, s), ¹³C-NMR (δ) (CDCl₃) C-1 (129.3), C-2 (165.6), C-3 (185.9), C-4 (124.0), C-5 (169.6), C-6 (33.0), C-7 (33.6), C-8 (34.6), C-9 (55.5), C-10 (46.5), C-11 (33.8), C-12 (78.7), C-13 (47.8), C-14 (47.9), C-15 (23.6), C-16 (29.8), C-17 (81.7), C-18. (6.0), C-19 (16.3), C-20 (23.5).

2α-Methyl-9α,17β-dihydroxy-5α-androstane (5)

White solid; melting point: 231-233° C.; [α]_{D}²⁵=−44.1 (c 0.001); IR υₘₐₓ (cm⁻¹): 3478 (O—H), 1652 (C═O); HRFAB-MS (+ve) m/z 303.2287 [M⁺] (calc. 303.2324) (C₂₀H₃₁O₃); FAB-MS (+ve) m/z 303.1; ¹H-NMR (δ) (d₆-acetone), H₂-1 (1.76, overlap; 1.65, overlap), H₂-2 (2.43, m) H₂-4 (2.22, overlap; 2.10, overlap), H-5 (2.18, overlap), H₂-6 (1.27, overlap, 2[H]), H₂-7 (1.75, overlap; 1.54, overlap), H-8 (1.77, overlap), H₂-11 (1.40, overlap, 2[H]), H₂-12 (1.64, overlap; 1.34, overlap), H-14 (1.43, overlap), H₂-15 (1.51, m; 1.23, overlap), H₂-16 (2.08 overlap; 1.41, overlap), H-17 (3.70, t J_{17,16=8.4} Hz), H₃-18 (0.77, s), (1.18, s), H₃-20 (1.00, d, J_{20,2}=6.4 Hz), ¹³C-NMR (δ) (d₆-acetone), C-1 (41.6), C-2 (40.9), C-3 (212.5), C-4 (44.7), (C-5 (39.8), C-6 (28.3), C-7 (27.3), C-8 (37.4), C-9 (75.5), C-10 (41.0), C-11 (24.9), C-12 (32.2), C-13 (43.0), C-14 (43.9), C-15 (23.2), C-16 (30.5), C-17 (81.5), C-18 (10.2), C-19 (14.3), C-19 (14.7).

6-Methylene-3α,7β,17βtrihydroxy-5β-androstane (7)

White solid; melting point: 241-243° C.; [α]_{D}²⁵=−88.3 (c 0.001); IR υₘₐₓ (cm⁻¹; 3398.7 (O—H), HRFAB-MS m/z 315.1949 [M⁺] (calc. 315.1960) (C₂₀H₂₇O ₃); FAB-MS (−ve) m/z 313.3; ¹H-NMR (δ) (CH₃OD), H-1 (1.53, overlap, 2[H]) H-2 (1.66_{;} overlap; 1.55, overlap), H-3 (4.09, br. s), H₂-4 (1.67, overlap, 2[H]), H-5 (2.83, overlap), H-7 (3.99, br. a), H-8 (1.50, overlap), H₂-11 (1.64, overlap; 1.27, overlap), H₂-12 (1.80, overlap; 1.06, overlap), H-14 (1.49, overlap), H₂-15 (1.75, m; 1.30, overlap), H₂-16 (2.01, overlap; 1.49, overlap), H-17 (3.63, t, J_{17,16}=8.4 Hz), H₃-18 (0.71, s), H₃-19 (0.67, s), H₂-20(4.86, s; 4.52, s), ¹³C-NMR (δ) (CH₃OD), C-1 (32.9), C-2 (31.8), C-3 (66.9), C-4 (29.1), C-5 (38.7), C-6 (1.54.), C-7 (75.0) C-8 (42.7), C-9 (46.0), C-10 (39.4), C-11 (21.7), C-12 (37.6), C-13 (44.0), C-14 (47.6), C-15 (23.5), C-16 (30.6), C-17 (82.5), C-18 (10.9), C-19 (11.3), C20 (107.9).

11α,17β-Dihydroxy-6-methylene-androsta-1,4-diene-3-one (8)

White solid; UV λₘₐₓ (log ε): 248 (5.81); melting point: 239-241° C.; [α]_{D}²⁵+134.0 (c 0.001); IR υₘₐₓ (cm⁻¹): 3431 (O—H), 1663 (C═O): HREI-MS m/z 320.2348 [M⁺] (calc, 320.2351) (C₂₀H₃₂O₃); EI-MS m/z (%): 320.2 [M⁺] (100), 302.3 (26), 249.2 (28), 168.1 (30): ¹H-NMR (δ) (CDCl₃) H-1 (7.88, J_{1,2}=10.1 Hz), H-2 (6.12, J_{2,1}=10.1 Hz), H-4 (6.13, s), H₂-7 (2.56, overlap; 1.86, overlap), H-8 (1.86, overlap), H-9 (1.33, overlap), H-11 (4.04, td, J_{a,a}=10.7 Hz; J_{a,e}=4.2. Hz), H₂-12 (2.12, overlap; 1.10, overlap), H-14 (1.13, overlap), H₂-15 (1.67, m; 1.31, overlap), H₂-16 (2.02, m; 1.49, overlap), H-17 (3.67, t, J_{17,16}=8.4 Hz), H₃-18 (0.80, s), H₃-19 (1.23, s), ¹³C-NMR (δ) (CDCl₃), C-1 (161.4), C-2 (124.7), C-3 (189.1), C-4 (123.1), C-5 (170.9), C-6 (147.8), C-7 (41.1). C-8 (36.2), C-9 (59.6), C-10 (44.2), C-11 (67.9), C-12 (48.3), C-13 (45.7), C-14 (50.8), C-15 (24.1), C-16 (30.7), C-17 (81.5), C-18 (12.4), C.-19 (20.2), C-20 (20.2).

### Human Placental Aromatase Inhibition Assay Protocol

Transformation of testosterone to 17β-estradiol (shown as follows) can be used for the measurement of aromatase enzyme activity).

The aromatase enzyme activity is determined in a 1 mL reaction mixture, containing 2 mg/mL aromatase enzyme, 10 μM testosterone, 0.1 M potassium phosphate buffer at pH 7.4, and 10 μL of test compound (0.1 mM). Mixture was pre-incubated for 10 minutes at 37° C. NADPH (1 mM) was added in the mixture, and incubated for 20 minutes. Trichloroacetic acid (10%, w/v) (100 μL) was added to stop the reaction, centrifuged at 12,000 g for 10 minutes, pellet was discarded, and the supernatant (17β-estradiol) was extracted with N-butyl chloride (1 mL), The extract (17β-estradiol) was dried, and the product quantity was determined by UPLC (column ACE Generix 5 μm C₁₈ 150×4.6 mm) using isocratic elution of the mobile phase containing triethylamine (0.1%) in ACN/H₂O (45.55, v/v), and pH 3.0 (adjusted by orthophosphoric acid) with a flow rate of 1.2 ml/min at 200 nm. Calculations were performed by following formula:

% Inhibition=100−(Peak area of test sample/Peak area of control)×100

### Results and Discussion

The HREI-MS of derivative 2 showed the [M]⁺ at m/z 314.1873 (calc. 314.1882) (C₂₀H₂₆O₃), indicating addition of an oxygen atom as a hydroxyl group atamestane (1) (m/z 300), Hydroxylation at C-11 was inferred on the basis of HMBC correlations of H-8, H-15, H₂-7, and H₂-12 with C-14. The structure of new compound was determined as 14α-hydroxy-1-methylandrosta-1,4-diene-3,17-dione (2).

Derivative 4 showed its [M+H]⁺ at in m/z 317.2124 (calc. 317.2117) (C₂₀Al₂₉O₃) in the HRFAB-MS (+ve mode). This suggested the cleavage of ester moiety at C-17, hydroxylation, and dehydrogenation in drostanolone enanthate (m/z 418.7). Hydroxylation at C-12 was inferred on the basis of HMBC correlations of H-8, H-9, and H₃-19 C-12. Dehydrogenation between C-1/C-2, and C-4/C-5 was determined by HMBC ²J interactions of H-4 with C-3 and C-5. and H-1 with C-10. The structure of 4 was determined as 2-methylandrosta-12β,17β-dihydroxy-1,4-diene-3-one.

The HRFAB-MS (+ve) of metabolite 5 m/z 303.2287 [M+H]⁺ (calc. 301.2324), indicating hydrolysis of ester group at C-17, along with hydroxylation in drostanolone enanthate (m/z 418.7). Presence of OH group at C-9 was inferred via the ²J correlations of H-8 and H₂-11 with C-9, and ³J interaction of H₃-19 with C-9 in the HMBC spectrum. The structure of 5 was deduced as 2α-methyl-9α,17β-dihydroxy-5α-androstane (7).

The [M+H]⁺ of compound 7 was observed at m/z 315.1949 (calc. 315.1960) (C₂₀H₂₇O₃) in the HRFAB-MS (+ve mode), indicating reduction of olefinic (C-1, C-2, C-4, and C-5), and ketonic carbons (C-3, and C-17), along with hydroxylation in exemestane (6) (m/z 296.1). The structure was determined as 6-methylene-3α,7β,17β-trihydroxy-5β-androstane (7).

Transformed product 8 displayed its [M]⁺ at m/z 320.2348 (calc. 320.2351) (C₂₀H₃₂O₃)317.2124 (calc. 317.2117) (C₂₀H₂₉O₃) in the HREI-MS. This suggested addition of an oxygen, and two hydrogen atoms in exemestane (6) (m/z 296.1). The structure was determined as 11α,17β-dihydroxy-6-methylene-androsta-1,4-diene-3-one (8),

Presence of an α-OH group at C-14 in metabolite 2 has increased its inhibition potential against human aromatase enzyme, as compared to the parent drag atamestane (1).

Presence of β-OH groups at C-12 and C-17, and dehydrogenation in the ring A of derivative 4 have enhanced its anti-aromatase activity, in comparison to the parent drug drostanolone enanthate (3), while presence of β-OH at C-17, and α-OH at C-9 in derivative 5 has decreased its activity.

βHydroxylation at C-17 and C-7, and a hydroxylation at C-3, along with hydrogenation in the ring A of compound 7 have increased its activity against human aromatase enzyme in contrast to parent drug exemestane (6), whereas presence of an α-OH at C-11, and β-OH at C-17 in compound 8 has decreased its activity.

What is claimed is:

1. A method of treatment of diseases associated with the over-expression of aromatase enzyme, including breast cancers, and male infertility, based on administration of effective amount of newly developed aromatase inhibitors having formulae 2, 4, 5, 7, and 8 or their isomers, salts or solvates, or co-crystals in suitable pharmaceutical excipients, adjuvant, carrier, or diluent to humans, and animals in need thereof.

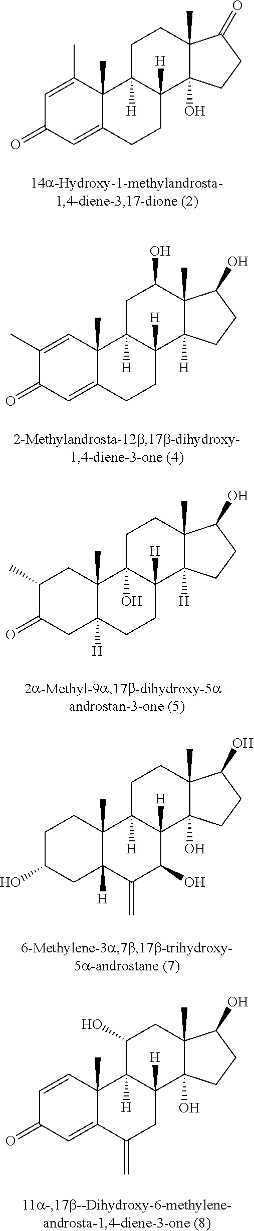

2. Formulae 2 (IC₅₀=9.7±0.72 nM), 4 (IC₅₀=4.23±0.133 nM), 5 (IC₅₀=793±29.9 nM), 7 (IC₅₀=46.1±0.81 nM), and 8 (IC₅₀=12797±844 nM) as in claim 1, are new steroidal-based potent aromatase inhibitors that reduces, inhibits, or abrogates activity of aromatase enzyme, and thereby can treat estrogen-responsive (ER+) breast cancer, and improving testosterone/estradiol (T/E) ratio levels in infertile male.

3. Formula 2 as in claim 1, can be synthesized by biotransformation of anti-cancer drug atamestane (1) or through the chemical synthesis.

4. Formulae 4, and 5 as in claim 1, can be synthesized by biotransformation of anti-cancer drug drostanolone enanthate (3) or through the chemical synthesis.

5. Formulae 7, and 8 as in claim 1, can be synthesized by biotransformation of anti-cancer drug exemestane (6) or through the chemical synthesis.

6. Formulae 2, 4, 5, 7, and 8 as in claim 1, can also be used for the prevention of other diseases resulted from the over-expression of aromatase enzyme.
